(19) United States
(12) Patent Application Publication
(10) Pub. No.: US 2006/0035811 A1
(43) Pub. Date: Feb. 16, 2006

(54) Methods of treating dry eye disease with lantibiotics

(76) Inventor: Luis Molina, Durham, NC (US)

Correspondence Address: KING & SPALDING LLP, 191 PEACHTREE STREET, N.E., 45TH FLOOR, ATLANTA, GA, 30303-1763, US

(21) Appl. No.: 10/531,660

(22) PCT Filed: Sep. 22, 2003
(86) PCT No.: PCT/US03/29853

§ 371 Date: Apr. 15, 2005

Related U.S. Application Data
(60) Provisional application No. 60/419,639, filed on Oct. 18, 2002.

(51) Int. Cl. A61K 38/16

(52) U.S. Cl. 514/2

(57) ABSTRACT

The treatment of dry eye disease with lantibiotics such as duramycin is described, along with pharmaceutical formulations useful for carrying out such treatments.

### RELATED APPLICATIONS

This application claims the benefit of U.S. Provisional Patent Application Ser. No. 60/419,639, filed Oct. 18, 2002, the disclosure of which is incorporated by reference herein in its entirety.

### FIELD OF THE INVENTION

The present invention concerns methods of treating dry eye disease, along with pharmaceutical compositions useful in such methods.

### BACKGROUND OF THE INVENTION

Dry eye disease is the general term for abnormalities of the precorneal tear film characterized by a decrease in tear production or an increase in tear film evaporation, together with the ocular surface disease that results. A variety of causes of dry eye disease are known, and approximately 38 million Americans are affected with some type of dry eye disease.

U.S. Pat. No. 6,277,855 to Yerxa describes methods of treating dry eye disease with nicotinic acetylcholine receptor agonists.

U.S. Pat. No. 5,900,407 to Yerxa et al. describes methods of treating dry eye disease with uridine triphosphates and related compounds.

U.S. Pat. No. 5,716,931 to Molina et al. describes methods of treating retained pulmonary secretions with lantibiotics such as duramycin

There remains a need for new ways to treat dry eye disease.

### SUMMARY OF THE INVENTION

A first aspect of the present invention is a method of treating dry eye disease in a subject, comprising administering to said subject a lantibiotic such as duramycin in an amount effective to treat dry eye disease. The lantibiotic is typically administered topically or systemically, and in a pharmaceutically acceptable carrier.

A further aspect of the present invention is the use of a lantibiotic as described herein for the preparation of a medicament for treating dry eye disease as described herein.

A still further aspect of the present invention is a pharmaceutical composition for treating dry eye disease as described herein comprising, consisting of or consisting essentially of a lantibiotic such as duramycin as described herein in a pharmaceutically acceptable carrier as described herein.

### DETAILED DESCRIPTION OF THE PREFERRED EMBODIMENTS

Subjects which may be treated by the methods of the present invention include both human subjects for medicinal purposes and animal subjects (e.g., dog, cat, rat, horse), particularly mammalian subjects, for veterinary or drug development purposes.

Any type of dry eye disease may be treated by the methods and compositions of the present invention, including but not limited to includes keratoconjunctivitis sicca (KCS), age-related dry eye, Stevens-Johnson syndrome, Sjogren's syndrome, ocular cicatrical pemphigoid, blepharitis, Riley-Day syndrome, and congenital alacrima. Dry eye disease can also be caused by nutritional disorders or deficiencies (including vitamins), pharmacologic side effects, eye stress and glandular and tissue destruction, environmental exposure to smog, smoke, excessively dry air, airborne particulates, autoimmune and other immunodeficient disorders, and comatose patients who are unable to blink.

Applicant specifically intends that all patent references cited herein be incorporated by reference herein in their entirety.

Lantibiotics that may be used to carry out the present invention include, but are not limited to, duramycin, nisin, subtilin (Gross et al. Z. Physiol. Chem., 354, 810 (1973)), epidermin (Schnell et al. Nature, 333,276 (1988)), Pep 5 (Sahl, J. Bacteriol., 162, 833 (1985)), gallidermin (Kellner et al, Eur. J. Biochem. 177, 53 (1988)). mersacidin, actagardine (Kettenring et al., J. Antibiotics, 53, 1082 (1990)), cinnamycin (Kessler et al., Helv. Chim. Acta, 71, 1924 (1988)), duramycin, and ancovenin (Wakamiya et al., Tetrahedron Lett 26, 665 (1985)). The lantibiotic can be naturally occurring or produced by genetic engineering techniques. Compounds such as these are known and can be made in accordance with known procedures, or variations thereof that will be apparent to those skilled in the art.

The structure of duramycin is known. See Hayashi et al., J. Antibiotics, 43, 1421 (1990). Duramycin is available from Sigma Chemical Co. (St. Louis, Mo., USA) as catalog no. D3168, or can be produced in accordance with known techniques from Streptoverticilium cinnamoneum subsp. azacolutum (NRRL B-1699) (available from the USDA Agricultural Research Service, Peoria, Ill., USA) in accordance with known techniques. See, e.g., Hayashi et al., supra, Pridham et al, Phytopathology 46, 575-581 (1956); Shotwell et al., J. Am. Chem. Soc. 80, 3912 (1958); S. Nakamura et al. Biochem 23,385 (1984).

The active compounds disclosed herein can, as noted above, be prepared in the form of their pharmaceutically acceptable salts. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, maleic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine, and (c) salts derived from bases, such as ammonium salts, alkali metal salts such as those of sodium and potassium, alkaline earth metal salts such as those of calcium and magnesium, and salts with organic bases such as dicyclohexylamine and N-methyl-D-glucamine.

Active agents described herein may be administered in like manner as different active agents that have been described for the treatment of dry eye disease, such as described in U.S. Pat. No. 6,277,855 to Yerxa, or U.S. Pat. No. 5,900,407 to Yerxa et al.

The active compounds disclosed herein may be administered topically or systemically. For topical application, the active compounds are administered to the eyes of a patient by any suitable means, but are preferably administered by a liquid or gel suspension of the active compound in the form of drops, spray or gel. Alternatively, the active compounds may be applied to the eye via liposomes. Further, the active compounds may be infused into the tear film via a pump-catheter system. Another embodiment of the present invention involves the active compound contained within a continuous or selective-release device, for example, membranes such as, but not limited to, those employed in the Ocusert.™ System (Alza Corp., Palo Alto, Calif.). As an additional embodiment, the active compounds can be contained within, carried by, or attached to contact lenses which are placed on the eye. Another embodiment of the present invention involves the active compound contained within a swab or sponge which can be applied to the ocular surface. Another embodiment of the present invention involves the active compound contained within a liquid spray which can be applied to the ocular surface.

The topical solution containing the active compound may contain a physiologically compatible vehicle, as those skilled in the ophthalmic art can select, using conventional criteria The vehicles may be selected from the known ophthalmic vehicles which include, but are not limited to, saline solution, water polyethers such as polyethylene glycol, polyvinyls such as polyvinyl alcohol and povidone, cellulose derivatives such as methylcellulose and hydroxypropyl methylcellulose, petroleum derivatives such as mineral oil and white petrolatum, animal fats such as lanolin, polymers of acrylic acid such as carboxypolymethylene gel, vegetable fats such as peanut oil and polysaccharides such as dextrans, and glycosaminoglycans such as sodium hyaluronate and salts such as sodium chloride and potassium chloride.

In addition to the topical method of administration described above, various methods can be used to administer the active compounds of the present invention systemically to eyes. The term systemic as used herein includes subcutaneous injection; intravenous, intramuscular, intrasternal injection; infusion; inhalation, transdermal administration, oral administration; and intra-operative instillation.

One systemic method involves an aerosol suspension of respirable particles comprising the active compound, which the subject inhales. The active compound would be absorbed into the bloodstream via the lungs, and subsequently contact the lacrimal glands in a pharmaceutically effective amount. The respirable particles may be liquid or solid, with a particle size sufficiently small to pass through the mouth and larynx upon inhalation; in general, particles ranging from about 1 to 10 microns, but more preferably 1-5 microns, in size are considered respirable.

Another method of systemically administering the active compounds to the eyes of the subject would involve administering a liquid/liquid suspension in the form of eye drops or eye wash or nasal drops of a liquid formulation, or a nasal spray of respirable particles which the subject inhales. Liquid pharmaceutical compositions of the active compound for producing a nasal spray or nasal or eye drops may be prepared by combining the active compound with a suitable vehicle, such as sterile pyrogen free water or sterile saline by techniques known to those skilled in the art.

Other methods of systemic administration of the active compound involves oral administration, in which pharmaceutical compositions containing active compounds are in the form of tablets, lozenges, aqueous or oily suspensions, viscous gels, chewable gums, dispersible powders or granules, emulsion, hard or soft capsules, or syrups or elixirs. Tablets contain the active ingredient in admixture with nontoxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil. Formulation for oral use may also be presented as chewable gums by embedding the active ingredient in gums so that the active ingredient is slowly released upon chewing.

Additional means of systemic administering of the active compound to the eyes of the subject would involve a suppository form of the active compound, such that a therapeutically effective amount of the compound reaches the eyes via systemic absorption and circulation.

Further means of systemic administration of the active compound involve direct intra-operative instillation of a gel, cream, or liquid suspension form of a therapeutically effective amount of the active compound.

For systemic administration such as injection and infusion, the pharmaceutical formulation is prepared in a sterile medium. The active ingredient, depending on the vehicle and concentration used, can either be suspended or dissolved in the vehicle. Adjuvants such as local anaesthetics, preservatives and buffering agents can also be dissolved in the vehicle. The sterile injectable preparation may be a sterile injectable solution or suspension in a non-toxic acceptable diluent or solvent. Among the acceptable vehicles and solvents that may be employed are sterile water, saline solution, or Ringer's solution.

For oral use, an aqueous suspension is prepared by addition of water to dispersible powders and granules with a dispersing or wetting agent, suspending agent one or more preservatives, and other excipients. Suspending agents include, for example, sodium carboxymethylcellulose, methylcellulose and sodium alginate. Dispersing or wetting agents include naturally-occurring phosphatides, condensation products of an allylene oxide with fatty acids, condensation products of ethylene oxide with long chain aliphatic alcohols, condensation products of ethylene oxide with partial esters from fatty acids and a hexitol, and condensation products of ethylene oxide with partial esters derived from fatty acids and hexitol anydrides. Preservatives include, for example, ethyl, and n-propyl p-hydroxybenzoate. Other excipients include sweetening agents (e.g., sucrose, saccharin), flavoring agents and coloring agents. Those skilled in the art will recognize the many specific excipients and wetting agents encompassed by the general description above.

For rectal administration, the compositions in the form of suppositories can be prepared by mixing the active ingredient with a suitable non-irritating excipient which is solid at ordinary temperatures but liquid at the rectal temperature and will therefore melt in the rectum to release the compound. Such excipients include cocoa butter and polyethylene glycols.

The appropriate dosage of the lantibiotic will depend on factors such as the particular lantibiotic, the condition of the subject, the route of administration, etc., but can be determined in accordance with known techniques. For example, for topical administration, the dosage may be from 0.1, 1 or 10 nanograms of lantibiotic per milliliter of carrier, up to 10 or 20 milligrams of lantibiotic per milliliter of carrier, or more.

Compositions of the present invention may include one or more additional active agents, such as nicotinic acetylcholine receptor agonists as described in U.S. Pat. No. 6,277,855 to Yerxa, and uridine triphosphates as described in U.S. Pat. No. 5,900,407 to Yerxa et al.

### EXAMPLE 1

### Topical Treatment of Dry Eye Disease with Duramycin

A pharmaceutical formulation is prepared consisting of sterile, pyrogen free, physiological saline solution as a carrier, and containing from 10 nanograms to 1 milligram of duramycin as an active agent An adult human subject afflicted with dry eye disease is administered several drops of the pharmaceutical formulation in each eye to ameiliorate the symptoms of the dry eye disease. Administration is repeated when desired by the subject for further relief, or to continue relief, of the symptoms of the dry eye disease.

### EXAMPLE 2

### Topical Application of Duramycin in Albino Rabbits

New Zealand (albino) rabbits were the experimental model used to test the action of duramycin. Four animals were involved for each formulation: Duramycin was used in a formulation at 0.5 mg/ml dissolved in saline (0.9% sodium chloride) at pH between 6 and 7 and was compared with 0.9% sodium chloride and with 0.9% sodium chloride at pH between 6 and 7 (vehicle). Animals were randomly allocated into 3 groups of 4 animals each by a computer controlled program.

Aliquots (1×50 microliters) were administered using a micropipette into the right eyes while the left eyes remained untreated. Sampling was performed in the right eyes for a constant interval of 15 seconds before the instillation and at 15, 30, 60, 120, and 180 minutes after instillation. Tear secretion was collected and measured on treated eyes using Schimer strips.

Tear secretion, measured as the tear migration onto Schirmer strips for 15 seconds, was expressed in millimeters (mm) and in percents (%) of baseline values. The % of baseline values was the mean of individual %. T max is the time when the maximum mean value is obtained and is expressed in minutes (min).

Mean AUC_{0-180 min} value (area under the curve from baseline to 180 min) is the mean of individual AUC_{0-180 min} values calculated using the trapezoidal rule and is expressed in mm×min and in %×min.

Results of the experiment are summarized in Table 1 below. Tmax was obtained after 30 min of instillation for duramycin and for vehicle and at time zero for saline. Baseline value (n=12) was 3.1±1.4 mm. Compared with the saline treatment the vehicle slightly increased tear secretion 1.33 and 2.43 times based on AUC_{0-180 min} values in mm and in % respectively and by 1.29 and 2.05 times based on the maximum values in mm and in % respectively. Duramycin clearly increased tear secretion by 1.92 and 3.56 times based on AUC_{0-180 min} values in mm and in % respectively and by 2.47 and 4.59 times based on the maximum values in mm and in % respectively.

The foregoing is illustrative of the present invention, and is not to be construed as limiting thereof. The invention is defined by the following claims, with equivalents of the claims to be included therein.

TABLE 1
Tear Secretion in Albino Rabbits

| | Saline | | | | Vehicle | | | | Duramycin 0.5 mg/ml | | | |
|---|---|---|---|---|---|---|---|---|---|---|---|---|
| Time after | Tear secretion | | Tear secretion | | Tear secretion | | Tear secretion | | Tear secretion | | Tear secretion | |
| instillation | (mm) | | % | | (mm) | | % | | (mm) | | % | |
| (min) | Mean | SD | Mean | SD | Mean | SD | Mean | SD | Mean | SD | Mean | SD |
| 0 | 3.8 | 1.3 | 100.0 | 0.0 | 2.8 | 1.7 | 100.0 | 0.0 | 2.8 | 1.3 | 100.0 | 0.0 |
| 15 | 3.5 | 1.3 | 100.0 | 20.4 | 4.3 | 2.5 | 155.8 | 65.2 | 6.0 | 2.8 | 329.2 | 326.7 |
| 30 | 2.8 | 1.5 | 81.3 | 37.5 | 4.9 | 1.7 | 205.0 | 61.4 | 9.4 | 2.5 | 459.4 | 370.7 |
| 60 | 3.0 | 2.2 | 75.0 | 54.0 | 4.0 | 0.8 | 195.8 | 137.7 | 4.8 | 1.4 | 260.4 | 262.8 |
| 120 | 2.3 | 1.9 | 62.5 | 43.3 | 2.9 | 2.3 | 158.8 | 151.0 | 3.5 | 0.7 | 157.3 | 95.4 |
| 180 | 1.8 | 2.4 | 43.8 | 59.1 | 2.4 | 1.4 | 144.2 | 145.0 | 4.9 | 3.9 | 245.8 | 213.2 |

| | | | | | | |
|---|---|---|---|---|---|---|
| AUC _{0-180 mm ×} | 465.0 | 12515.6 | 617.8 | 30362.5 | 891.6 | 44554.7 |
| _{min % × min} | | | | | | |

That which is claimed is:

1. A method of treating dry eye disease comprising administering to a subject in need of such treatment a therapeutically effective amount of a lantibiotic in a pharmaceutically effective carrier.

2. The method according to claim 1, wherein said lantibiotic is duramycin.

3. The method according to claim 1, wherein said administering involves topical administration of said compound.

4. The method according to claim 3, wherein said topical administration is via a carrier vehicle selected from a group consisting of drops of liquid, liquid washes, gels, ointments, sprays an liposomes.

5. The method according to claim 3, wherein said topical administration comprises infusion of said compound to said ocular surface via a device selected from a group consisting of a pump-catheter system, a continuous or selective release device and a contact lens.

6. The method according to claim 1, wherein said administering is systemic administration of said compound.

7. The method according to claim 6, wherein said systemic administration involves administration of a liquid/liquid suspension of said compound via nose drops or nasal spray, or administration of a nebulized liquid to oral or nasopharyngeal airways of said subject, such that a therapeutically effective amount of said compound contacts lacrimal tissues of said subject via systemic absorption and circulation.

8. The method according to claim 6, wherein said systemic administration of said compound is accomplished by administering an oral form of said compound, such that a therapeutically effective amount of said compound contacts lacrimal tissues of said subject via systemic absorption and circulation.

9. The method according to claim 6, wherein said systemic administration involves administration of an injectable form of said compound, such that a therapeutically effective amount of said compound contacts lacrimal tissues of said subject via systemic absorption and circulation.

10. The method according to claim 6, wherein said systemic administration involves administration of a suppository form of said compound, such that a therapeutically effective amount of said compound contacts lacrimal tissues of said subject via systemic absorption and circulation.

11. The method according to claim 6, wherein said systemic administration involves administration of an intra-operative instillation of a gel, cream, powder, foam, crystals, liposomes, spray or liquid suspension form of said compound, such that a therapeutically effective amount of said compound contacts the lacrimal tissues of said subject via systemic absorption and circulation.
